(19) United States
(12) Patent Application Publication
(10) Pub. No.: US 2015/0056333 A1
(43) Pub. Date: Feb. 26, 2015

(54) NANOTECHNOLOGY SMART STICKER FOR USE WITH PERISHABLE FOODS

(71) Applicants: Sinday Johanna Chaves Noguera, Heredia (CR); Juan Scott Chaves Noguera, Heredia (CR)

(72) Inventors: Sinday Johanna Chaves Noguera, Heredia (CR); Juan Scott Chaves Noguera, Heredia (CR)

(73) Assignee: Etipres, SA, Heredia (CR)

(21) Appl. No.: 14/010,368

(22) Filed: Aug. 26, 2013

(51) Int. Cl. G01N 33/02

(52) U.S. Cl. CPC G01N 33/02
USPC 426/87

(57) ABSTRACT

A “smart sticker” employing nanotechnology for determining the current state of raw meat for safe human consumption is provided. The sticker indicates the quality to the consumer by providing a color changing indicator on the sticker. The sticker employs carbon nanoparticles containing iron, which are placed on an underside portion of the sticker in contact with the raw meat. The nanoparticles serve as “bait” for the harmful bacteria. When pathogenic bacteria sense the presence of iron, it produces certain molecules to sequester the iron. Those molecules move to the area where the iron is present in the sticker, which can then determine the amount of pathogenic bacteria present in the meat by way of a chemical reaction with a particular ink containing a novel additive, which reacts with the molecules secreted by the pathogens. Once this interaction occurs, the ink changes color from, for example, blue to yellow.

### FIELD OF THE INVENTION

The present invention relates to smart stickers for use with perishable foods to indicate freshness and the presence of harmful bacteria. More particularly, it relates to a nanotechnology smart sticker for use with raw meats, such as poultry, beef, pork, fish, among others, which has a novel additive applied thereupon, and which changes color once it detects the presence of harmful bacteria, of which is a result of the decaying process of raw meats over time since slaughter.

### BACKGROUND OF THE INVENTION

Each year, millions of people around the world get very sick or die due to bacterial food poisoning. Almost always, the people getting sick do not realize that they are consuming a perishable food that is tainted with harmful bacteria. Further, this problem is not regulated to the developing world, though statistics and probability of sickness are higher in such places. Rampant food poisoning from harmful bacteria is also widespread in the developed world and is occurring with alarming frequency. Bacteria such as Salmonella, Escherichia coli (also known as E. coli), as well as the larger genus bacteria Campylobacter (which includes E. Coli) are appearing in foods and making people sick around the world, including gastrointestinal flu, septicemia and even death in some cases from a severe bacterial infection.

Problematic food poisoning is exacerbated by the fact that consumers in all parts of the world are not aware that the perishable foods that they are buying to consume may contain any of the aforementioned or other harmful bacteria, which cause sickness and disease. And, the “Sell by Dates” and “Expirations Dates,” the most commonly used systems to indicate food freshness, are clearly inadequate in today's world where raw meat products from one part of the world are shipped to all other parts of the world by trains, trucks, ships and air, and where these products have a presumed expectation of freshness with an absence of harmful bacteria.

Smart stickers, also known as intelligent labels, for use with perishable foods are known in the prior art. Most work by employing a sticker on the perishable food wherein an area on the sticker changes color if a change of temperature has occurred. It is generally known that changes in temperatures can harm perishable foods, especially meat products. This can then result in sickness, and sometimes death, in people who consume these tainted products after the products have changed temperature or been exposed to varying temperatures, do to the presence of harmful bacteria that grows after said change in temperature has occurred. However, these stickers are most often used in a laboratory setting to determine if incubators, refrigerators and/or freezers are operating under normal conditions. These known prior art stickers do not provide an adequate system for the average consumer at a grocery store to make a determination if the raw meat that they are purchasing is safe to consume.

U.S. Pat. No. 5,254,473 to Patel discloses an example of such a smart sticker wherein a solid state device for monitoring integral values of time and temperature of storage of perishable foods, among other things, pharmaceutical products and other temperature sensitive products, such as perishable foods is provided. The device has an indicator composition capable of changing color in relationship to its exposure to a temperature above and below a pre-determined base line temperature and to the time of said exposure, for monitoring the time-temperature history of a substrate.

However, temperature change is not the only problem in the handling of perishable foods and in particular, raw meat. A host of other events or factors can occur which make the meat dangerous for human consumption because of the presence of harmful and pathogenic bacteria. For example, the improper handling (i.e., unclean slaughterhouse conditions, exposure to unclean human hands of the meat handlers, unsanitary packing procedures, to name just a few) of raw meat can cause harmful bacteria to be present, even when proper temperature handling of the meat has been observed. And, if any of these events occurred, but proper temperature handling was observed, then these temperature changing devices in the prior art would prove absolutely useless for the indication that harmful bacteria exists on the perishable meat products.

Accordingly, what is needed is an improved smart sticker that can be applied directly to raw meat of all types, as set forth above, for observance by handlers, packagers and consumers to indicate whether said raw meat contains a certain level of harmful bacteria, which if consumed is predisposed to cause food poisoning sickness in the average human being and which is not conditioned on whether there has been a change in temperature during any of the handling and packaging steps. The benefits of such improved smart sticker would be not only by released by the average consumer in the grocery store, but by hospitals, large food processing plants, food transportation companies, the hospitality industry and the health industry as a whole.

Reducing sickness from food poisoning has the benefit of reducing costs to socialized medical systems by reducing hospital stays and the unnecessary use of antibiotics, which are known to be over-prescribed. Likewise, in non-socialized medical systems, the increase in savings by insurance companies will help them use their financial resources to treat other currently non-preventable diseases, such as many of the various cancers. Either way, money will be saved in both medical care systems.

Such an improved smart sticker should employ a color changing sticker so that the average person can make an intelligent and informed choice by simple observance of the sticker in the grocery store, for example. Further, such improved smart sticker should employ nanotechnology to take advantage of working at the molecular or atomic level and to use this important area of science that uses such little natural resources, manpower, money and land to be produced.

### SUMMARY OF THE INVENTION

We have invented an improved “smart sticker” employing nanotechnology for determining the current state (i.e., the “quality”) of raw meats, including, but not limited to, beef, poultry, pork and fish, for safe human consumption. Our improved smart sticker indicates the quality to the consumer by providing a color changing indicator on a particular area of the sticker. The smart sticker can indicate to the customer two things: (1) if the appropriate temperature to handle the meat was compromised, and more importantly (2) if the presence of any harmful bacteria has increased to levels, which is considered harmful for human consumption, regardless of whether acceptable temperature thresholds have been exceeded. Our smart sticker is not dependent on a change in temperature to indicate the presence of harmful bacteria as with the prior art devices.

Instead, our improved smart sticker works by employing carbon encased nanoparticles containing iron, which are placed on a specific area of the sticker (typically on the underside, which is in direct contact with the raw meat). These nanoparticles, having the embedded iron, serve as “bait” for the harmful bacteria. When bacteria sense the presence of iron, it will start to produce and secrete certain molecules to sequester the iron. Those molecules move to the area where the iron is present (i.e., “bait on the sticker”). The number of secreted molecules is directly proportional to the number of pathogenic bacteria present. The sticker can then determine the amount of pathogenic bacteria present in the meat by a chemical reaction. In particular, once those molecules sequester the iron, a change of color will occur due to the fact that the area on the sticker where the bait is located contains a particular ink containing a novel additive, which reacts with the molecules secreted by the pathogens. Once this interaction occurs, the ink changes color from, for example, blue to yellow. The amount of bacteria considered to be “safe” for human consumption is determined based upon USDA standards and this determines the amount of additive to employ so that an accurate reading is provided by the person viewing the sticker.

It should be further noted that our improved smart sticker can be used with other perishable consumable foods besides meat. These other consumable perishable foods include, but are not limited to, fruits, vegetables, cheese and other dairy products and bread products or any other consumable perishable food wherein bacteria and/or mold could grow over time as a result of improper handling or time from slaughter and/or harvesting. It should be also noted that our improved smart sticker will help to detect harmful bacteria that occurs on foods where such bacteria is typically not grown, such as in the recent discoveries of E. Coli on cantaloupes and spinach.

### BRIEF DESCRIPTION OF THE DRAWINGS

The detailed description of the invention, contained herein below, may be better understood when accompanied by a brief description of the drawings, wherein:

FIG. 1 is a front side perspective view of a piece of raw meat with the smart sticker of the present invention applied thereupon;

FIG. 2 is a back side perspective view of the smart sticker showing the area where the ink and novel additive reside along with the nanoparticle embedded iron particles;

FIG. 3 is a cross-sectional view of the smart sticker along lines 3-3 of FIG. 2; and

FIG. 4 is an illustration of harmful bacteria on a piece of raw meat secreting molecules in search of iron particles to sequester for continued growth of the bacteria.

### DETAILED DESCRIPTION OF THE PREFERRED EMBODIMENT

Throughout the following detailed description, the same reference numerals refer to the same elements in all figures.

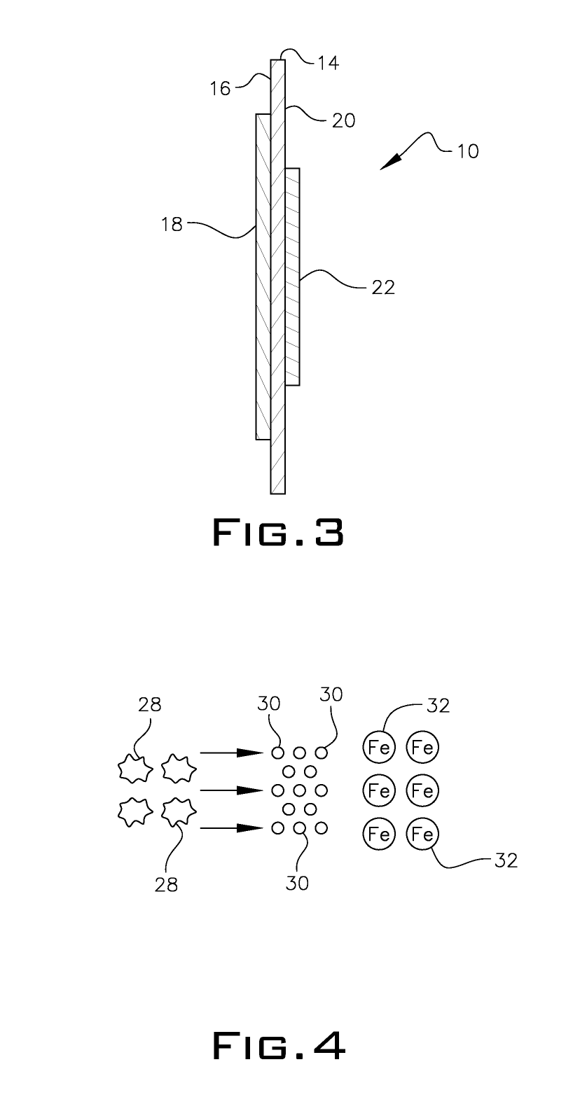

Referring to FIG. 1, a smart sticker 10 of the present invention is shown applied to a piece of raw meat 12. Sticker 10 has a substrate 14 (see FIG. 3), which has a top surface 16. An indicator window 18 is applied to the substrate top surface 16. Indicator window 18 is visible by the naked human eye to any person viewing sticker 10, to be described in more detail herein below.

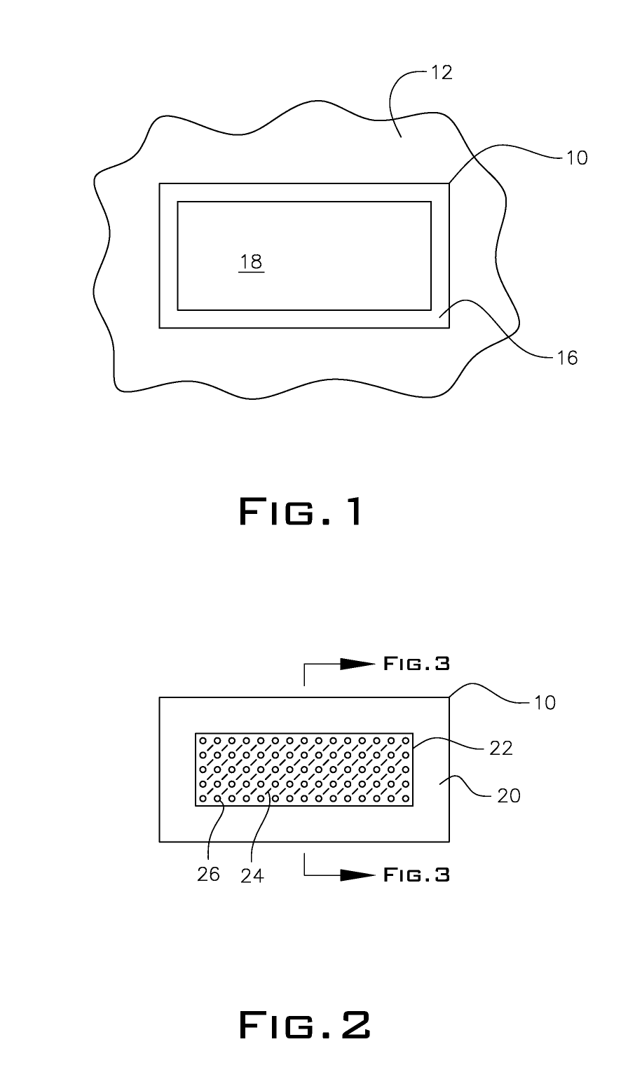

Referring to FIG. 2., a bottom surface 20 of substrate 14 (see also FIG. 3) has a containment area 22 for holding an ink 24 in which embedded therein is a plurality of nanoparticles 26 containing iron (FE) and an additive mixed within ink 24 made up the following chemical composition: 63% Chromeazurol S, a reagent to indicate or bring about a chemical reaction, 37% Hexadecyltrimethylammonium bromide, a cationic surfactant used as a detergent, 0.05% Iron(III) chloride, also referred to as ferric chloride, 0.005% carbon nanoparticles. The additive itself is made up of, in the preferred embodiment, 12% Chromeazurol S, 7% Hexadecyltrimethylammonium bromide, 0.01% Iron(III) chloride and 0.001% carbon nanoparticles. When combined with ink, the remaining compound is 80.99% of an acrylated oligomer.

Referring to FIG. 3, a cross-sectional view along lines 3-3 of FIG. 2, shows sticker 10 with indicator window 18 applied to top surface 16 of substrate 14 and containment area 22 applied to bottom surface 20 of substrate 14. Containment area 22 is the part of sticker 10 that is in direct contact with the raw meat of other perishable consumable food product. Although this is not directly illustrated in the figures, it is understood by appreciating the cross-sectional view of FIG. 2 and when considering FIG. 1 showing sticker 10 applied to raw meat 12, that containment area 22 is in direct contact with said raw meat 12.

Referring to FIG. 4, it is illustrated that bacteria 28 secrete molecules 30 in search of iron 32, also represented by its Periodic Table symbol FE. Iron is an essential nutrient mineral for bacteria growth and so sticker 10 uses it in the present invention to act as “bait” for the bacterial secreted molecules 30. When the bacterial secreted molecules 30 scavenge for this important nutrient, they come into contact with the iron 32 encased within nanoparticles 26, whereby a chemical reaction occurs that causes ink 24 to change color, from blue to yellow, for example, in the preferred embodiment. Nothing herein however requires that the change of color be regulated to just blue to yellow. Any other changing color combination can be employed.

The change in color of ink 24 in containment area 22 can then be seen by human observation with the naked eye in indicator window 18. Accordingly, it is understood that substrate 14 is not a “solid” barrier in between containment area 22 and indicator window 18 such that the chemical reaction and change in color of ink 24 occurring in containment area 22 on bottom surface 20 of substrate 14 cannot be seen in indicator window 18 applied to top surface 16 of substrate 14. It is just the opposite. Whatever change in color that occurs in ink 24 in containment area 22 can be observed by the naked human eye in indicator window 18 due to containment area 22 being in fluid communication with indicator window 18.

In the preferred embodiment, the additive using nanoparticles 26 is added to ink 24 and then printed onto sticker 10 in containment area 22, which is the part of sticker 10 that is direct contact with raw meat 12 or other perishable consumable food product and which is more porous than the other parts of sticker 10, thereby allowing ink 24 to be absorbed there within.

Iron 32, within the additive, are trapped around carbon nanoparticles 26. The quantity of nanoparticles employed is extremely small and in the preferred embodiment is in the range of 0.001%, which is just enough to allow bacteria 28 to sense its presence. Once the bacterial secreted molecules 30 reach iron 32, the chemical reaction occurs within the additive causing the change in color of ink 24. The ink color change is directionally proportional to the number of bacterial secreted molecules 30 it detects. This is because typically, a pathogen will secrete a molecule in search of iron of which when it finds it, will return “home” to deliver this essential nutrient to the pathogen and then return to sequester more iron. Therefore, the number of molecules detected by the additive is directly proportional to the pathogenic bacteria load. In other words, the rapidness in which the change of color of sticker 10 occurs indicates the amount of bacteria present from very small and therefore slightly dangerous to your health, to very large and therefore extremely dangerous to your health. Being able to determine this bacterial load is something not seen or taught anywhere in the prior art and is what makes sticker 10 of the present invention unique, novel and non-obvious.

Although not shown, the amount of bacterial load can be illustrated on sticker 10 by the depth of the color change or by using any number of icons to indicate the amount of bacteria 28 that is presence. Examples of icons that can be used instead of a simple color change include a thumbs up and thumbs down icon or a smiley or not smiley face icon, just to name two examples from the plurality of available icons that can be employed with the novel present invention.

Equivalent elements as described hereinabove can be substituted for the ones set forth herein to achieve the same results in the same manner and in the same way.

1. A smart sticker applied directly to a surface of perishable foods intended for human consumption used to indicate the presence of pathogenic bacteria on the surface of the perishable foods, the smart sticker comprising;
a) a substrate having a top and a bottom surface;
b) an indicator window applied to the substrate top surface;
c) a porous containment area applied to the substrate bottom surface such that the containment area is in fluid communication with the indicator window;
d) an ink applied to the porous containment area;
e) carbon nanoparticles with iron encapsulated there within and mixed with the ink; and
f) a chemical reactive additive mixed with the ink and the carbon nanoparticles with iron encapsulated there within which changes the color of the ink when the iron is sequestered by molecules secreted by the pathogenic bacteria located on the perishable food.

2. The smart sticker according to claim 1, wherein the indicator window and the porous containment area have a smaller surface area than the substrate.

3. The smart sticker according to claim 2, wherein the porous containment area has a smaller surface area than the indicator window.

4. The smart sticker according to claim 1, wherein the indicator window is transparent.

5. The smart sticker according to claim 1, wherein the ink, the carbon nanoparticles with iron encapsulated there within, and the chemical reactive additive are printed onto the smart sticker porous containment area.

5. (canceled)

6. The smart sticker according to claim 17, wherein the meat includes beef, poultry, pork and fish.

7. The smart sticker accordingly to claim 1, wherein the chemical reactive additive is formulated from Chromeazurol S, Hexadecyltrimethylammonium bromide, Iron(III) chloride and carbon nanocapsules.

8. The smart sticker according to claim 7, wherein the chemical reactive additive comprises approximately:
a) 63% Chromeazurol S by weight;
b) 37% Hexadecyltrimethylammonium bromide by weight;
c) 0.05% Iron(III) chloride by weight; and
d) 0.005% carbon nanocapsules by weight.

9. The smart sticker accordingly to claim 1, wherein the color-change is from blue to yellow.

10. The smart sticker according to claim 1, wherein the number of secreted molecules that have reacted with the carbon nanoparticles with iron encapsulated there within on the smart sticker determines a bacterial load of the bacteria present on the perishable food.

11. The smart sticker according to claim 7, wherein the ink and the chemical reactive additive combination comprises approximately:
a) 12% Chromeazurol S by weight;
b) 7% Hexadecyltrimethylammonium bromide by weight;
c) 0.01% Iron(III) chloride by weight;
d) 0.001% carbon nanocapsules by weight; and
e) 80.99% of an acrylated oligomer by weight.

12. A smart sticker applied directly to a surface of raw meat used to indicate the presence of bacteria on the surface of the raw meat, the smart sticker comprising;
a) a substrate having a top and bottom surface;
b) a transparent indicator window applied to the substrate top surface and having a smaller surface area than the substrate;
c) a porous containment area applied to the substrate bottom surface such that the containment area is in fluid communication with the indicator window, the porous containment area having a smaller surface area than the transparent indicator window;
d) an ink applied to the porous containment area;
e) carbon nanoparticles with iron encapsulated there within and mixed with the ink; and
f) a chemical reactive additive mixed with the ink and the carbon nanoparticles with iron encapsulated there within which changes the color of the ink when the iron is sequestered by molecules secreted by the bacteria located on the raw meat, the additive formulated from Chromeazurol S, Hexadecyltrimethylammonium bromide, Iron(III) chloride, carbon nanocapsules, and an acrylated oligomer.

13. The smart sticker according to claim 12, wherein the chemical reactive additive comprises approximately:
a) 63% Chromeazurol S by weight;
b) 37% Hexadecyltrimethylammonium bromide by weight;
c) 0.05% Iron(III) chloride by weight; and
d) 0.005% carbon nanocapsules by weight.

14. The smart sticker according to claim 12, wherein the ink, the carbon nanoparticles with iron encapsulated there within and the chemical reactive additive are printed onto the smart sticker porous containment area.

15. The smart sticker according to claim 12, wherein the color-change is from blue to yellow.

16. The smart sticker according to claim 12, wherein the ink and the chemical reactive additive combination comprises approximately:
a) 12% Chromeazurol S by weight;
b) 7% Hexadecyltrimethylammonium bromide by weight;
c) 0.01% Iron(III) chloride by weight;
d) 0.001% carbon nanocapsules by weight; and
e) 80.99% of an acrylated oligomer by weight.

17. The smart sticker according to claim 1, wherein the perishable foods include, meat, fruits, vegetables, cheese and other dairy products and bread products.
